(19) United States
(12) Patent Application Publication
(10) Pub. No.: US 2006/0212056 A1
(43) Pub. Date: Sep. 21, 2006

(54) Surgical instrument

(76) Inventors: Larry Salvadori, San Diego, CA (US); Lee Gour, La Mesa, CA (US); Martin W. Kerber, Sanford, FL (US)

Correspondence Address: BROWN, RUDNICK, BERLACK & ISRAELS, LLP., BOX IP, 18TH FLOOR, ONE FINANCIAL CENTER, BOSTON, MA, 02111, US

(21) Appl. No.: 10/536,545

(22) PCT Filed: Aug. 20, 2004
(86) PCT No.: PCT/US04/27011

§ 371 Date: Nov. 7, 2005

(30) Foreign Application Priority Data
Aug. 21, 2003 (US) 60496793

(51) Int. Cl. A61B 17/32

(52) U.S. Cl. 606/167

(57) ABSTRACT

A surgical instrument (10) is provided that includes a handle (18) defining a longitudinal axis (20). The handle has an outer surface (22) including a plurality of longitudinal fins (24) that define a plurality of longitudinal grooves (26) therebetween. At least one of the longitudinal fins may project radially from the outer surface of the handle. A pair of the fins may project radially from the outer surface of the handle and are diametrically opposed. A pair of the fins may be opposed and disposed in a plane tangential to the outer surface of the handle. Two separate pairs of the fins can project radially from the outer surface of the handle and are diametrically disposed. The two separate pairs are offset 90° relative to the longitudinal axis. Two separate pairs of the fins can be opposed and disposed in alternate planes tangential to the outer surface of the handle.

### BACKGROUND

1. Technical Field

The present disclosure generally relates to medical devices and, more particularly, to surgical instruments for suction, irrigation, etc. having improved manipulation features.

2. Description of the Related Art

Hand held medical devices are commonly used in surgical and dental procedures to aspirate fluids, irrigate, etc. adjacent a surgical site. Suction devices are used, for example, to remove saliva during dental procedures and to remove blood and other body fluids from surgical areas and thereby enhance the visibility of the surgical area. Certain surgical procedures require very precise placement and handling of suction devices and control of the vacuum pressure to avoid injury to a patient.

Suction devices have been designed with fine tips for accurate placement and with control vents for adjustment of vacuum pressure. One well known medical suction device is the Yankauer suction handle. The Yankauer suction handle includes an attachment point for connection to a vacuum source and a flexible distal tip that can be formed into an optimally curved shape and retain that shape during use. Certain versions of the Yankauer suction handle include vents for controlling vacuum pressure at the tip. A widened section of the Yankauer suction handle provides a gripping area for holding and manipulating the suction device.

The gripping area of a suction device can become very slippery when it is covered with bodily fluids. This is a serious drawback because suction devices are frequently used in an environment where they are immersed or splashed with bodily fluids or held by a medical practitioner whose hands are covered in bodily fluids. A slippery handle can cause a practitioner to drop or mishandle the suction device. Any such mishandling in the proximity of a sensitive area, such as, for example during neurosurgery, can cause grave injury to a patient.

Attempts have been made to overcome the drawbacks of the prior art, such as handles with transverse serrations. See, for example, U.S. Pat. No. 4,813,926. These type devices, however, may disadvantageously collect bodily fluids and cause the handle to become more slippery.

Therefore, it would be desirable to provide a surgical instrument having a handle that facilitates grip force and control of the instrument during a procedure, including those where bodily fluids are present. The handle may be configured to guide the flow of fluid to prevent pooling. It would be highly desirable if the handle is easily and efficiently manufactured and assembled.

### SUMMARY

Accordingly, a surgical instrument having a handle is provided that facilitates grip force and control of the instrument during a procedure, including those where bodily fluids are present to overcome the disadvantages and drawbacks of the prior art. The handle may be configured to guide the flow of fluid to prevent pooling. Most desirably, the handle is easily and efficiently manufactured and assembled.

In one particular embodiment, a surgical instrument is provided, in accordance with the principles of the present disclosure. The surgical instrument includes a handle defining a longitudinal axis. The handle has an outer surface including a plurality of longitudinal fins that define a plurality of longitudinal grooves therebetween. At least one of the longitudinal fins may project radially from the outer surface of the handle. A pair of the fins may project radially from the outer surface of the handle and are diametrically opposed. A pair of the fins may be opposed and disposed in a plane tangential to the outer surface of the handle. Two separate pairs of the fins can project radially from the outer surface of the handle and are diametrically disposed. The two separate pairs are offset 90° relative to the longitudinal axis. Two separate pairs of the fins can be opposed and disposed in alternate planes tangential to the outer surface of the handle.

The grooves of the handle may include guide channels that direct fluid to a proximal end of the handle. At least one of the grooves may define a greater volume than an adjacent groove.

In an alternate embodiment, the surgical instrument has an elongated tubular portion including an opening configured for suction. The tubular portion may include a passageway that extends to the opening having a nozzle. The tubular portion can have a curvature adjacent a distal portion thereof. A proximal end of the handle may have an attachment configured to communicate with a suction source.

The present disclosure overcomes the drawbacks of the prior art by providing a surgical instrument handle that prevents loss of control and slipping due to fluids and other debris. The handle made according to the present disclosure prevents mishandling of medical instruments and reduces patient injury, due at least in part to the configuration of the handle that provides grooves that channel fluids away from the gripping area of the handle.

Surgical instruments according to the present disclosure are readily manufacturable in large quantities using common materials such as thermoplastics and manufacturing techniques such as injection molding. The instruments according to the present disclosure can be cost effectively made using less material than surgical handles of the prior art that have, for example, substantially solid handles. Removal of mass from the handle to provide channels according to the present disclosure also advantageously shifts the center of mass of the surgical instrument toward the distal end thereof. A more distal center of mass facilitates control of the instrument by the practitioner and thereby reduces risks to patients.

In another alternate embodiment, the handle has a first wall thickness that smoothly increases to a second wall thickness. The first wall thickness and the second wall thickness may define a distal to proximal slope. The handle can have a slope configuration that defines a distal to proximal flow direction. Alternatively, the fins may radially extend from the handle and are spaced apart to facilitate rotational control. The fins can be configured to facilitate gripping. Each of the fins may have a thickness in the range of 0.060-0.065 inches.

In yet another alternate embodiment, a medical suction apparatus is provided that includes an elongated tubular portion having a passageway that extends to an opening having a nozzle that is configured for suction. A handle is mounted with the tubular portion and defines a longitudinal axis. The handle has an outer surface including a plurality of longitudinal fins. The plurality of longitudinal fins includes a first and second pair of the fins that project radially from the outer surface. The fins of each pair are diametrically opposed. The first and second pairs are offset 90° relative to the longitudinal axis of the handle. The plurality of longitudinal fins further includes a third and fourth separate pair of the fins that are opposed and disposed in alternate planes tangential to the outer surface of the handle. The plurality of longitudinal fins define a plurality of longitudinal grooves therebetween. The grooves include guide channels that direct fluid to a proximal end of the handle.

### BRIEF DESCRIPTION OF THE DRAWINGS

The objects and features of the present disclosure, which are believed to be novel, are set forth with particularity in the appended claims. The present disclosure, both as to its organization and manner of operation, together with further objectives and advantages, may be best understood by reference to the following description, taken in connection with the accompanying drawings wherein:

FIG. 1 is a perspective view of a surgical instrument in accordance with the principals of the present disclosure;

FIG. 2 is a side cutaway view of the surgical instrument shown in FIG. 1;

FIG. 3 is a side view of the surgical instrument shown in FIG. 1;

FIG. 4 is a cross sectional view of a handle of the surgical instrument taken along lines 4-4 of FIG. 3; and

FIG. 5 is a cross sectional view of the handle taken along lines 5-5 of FIG. 3.

### DETAILED DESCRIPTION OF THE EXEMPLARY EMBODIMENTS

The exemplary embodiments of the surgical instrument and methods of use disclosed are discussed in terms of a medical device and, more particularly, surgical instruments for suction, irrigation, etc. having improved manipulation features. These manipulation features advantageously facilitate gripping and control during a procedure, including those where bodily fluids are present, and are configured to guide the flow of fluid to prevent pooling. The surgical instrument may be used to withdraw fluids from a body cavity of a subject, for example, the surgical instrument may include a Yankauer type medical suction device having a flexible tip to facilitate accurate placement of suction for efficient removal of bodily fluids from a surgical area. The surgical instrument may also be used for applications requiring irrigation, pressurized air, etc. It is envisioned that the present disclosure may be employed with a range of medical devices including scalpels, clamps, spreaders, catheters, drills, picks, needle and syringe devices, mirrors, laser devices and the like. It is further envisioned that the surgical instrument may be employed for various medical applications such as, for example, dental, surgical, diagnostic, treatment, etc.

In the discussion that follows, the term “proximal” will refer to the portion of a structure that is closer to a practitioner, while the term “distal” will refer to the portion that is further from the practitioner. As used herein, the term “subject” refers to a human patient or other animal. According to the present disclosure, the term “practitioner” refers to a doctor, nurse, or other care provider and may include support personnel.

The following discussion includes a description of a surgical instrument in accordance with the principals of the present disclosure. Reference will now be made to the exemplary embodiments of the disclosure, which are illustrated in the accompanying figures.

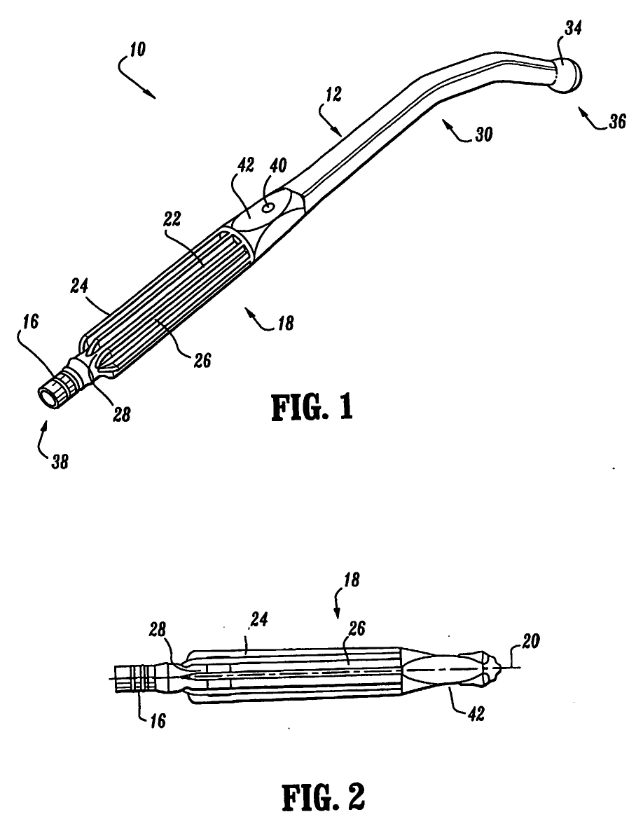

Turning now to the figures, wherein like components are designated by like reference numerals throughout the several views. Referring initially to FIGS. 1-3, a surgical instrument, such as, for example, a medical suction apparatus 10 includes an elongated tubular portion 12 having a passageway 14 that extends proximally to an opening having a nozzle 16 that is configured for suction. It is contemplated that nozzle 16 may be configured for irrigation, pressurized air and other procedures.

Passageway 14 extends through tubular portion 12 from a suction tip 34 at a distal end 36 of apparatus 10 to nozzle 16 at proximal end 38. Nozzle 16 is configured for connection to a vacuum source such as a vacuum pump. Vacuum is provided to suction tip 34 according to the negative pressure connected to nozzle 16. Tubular portion 12 has a substantially rectangular cross-section, with rounded surfaces, and may be variously dimensioned according to the requirements of a particular application. It is envisioned that tubular portion 12 may have various cross-sectional configurations, such as, for example, cylindrical, elliptical, polygonal, etc. Tip 34 may alternatively be configured for irrigation, pressurized air, etc.

The distal portion of tubular portion 12 has a curvature and includes a flexible portion 30, similar to a Yankauer type apparatus, to facilitate access for collection of fluids. Flexible portion 30 provides a range of flexibility 32 to facilitate desired placement for the collection of fluids. Flexible portion 30 facilitates placement so that a practitioner can manipulate suction apparatus 10 and not interfere with access or visibility to the surgical area. It is contemplated that, in an alternative embodiment, tubular portion 12 may be non-flexible. For example, tubular portion 12 may be semi-rigid, rigid, etc.

A handle 18 is mounted with tubular portion 12 and defines a longitudinal axis 20. Handle 18 has a substantially cylindrical cross-section and may be variously dimensioned according to the requirements of a particular application. It is further envisioned that handle 18 may have various cross-sectional configurations, such as, for example, cylindrical, rectangular, elliptical, polygonal, etc.

Handle 18 has a finger notch 42 that facilitates manipulation of apparatus 10. Finger notch 42 includes a depression that receives the thumb and forefinger of a practitioner. A vent hole 40 formed in finger notch 42 provides a vacuum/pressure channel from the ambient environment to passageway 14. Vent hole 42 can be open, partially covered or completely covered by a practitioner's finger or thumb to increase or reduce vacuum at suction tip 34. It is envisioned that finger notch 42 may have various configurations that enable manipulation of apparatus 10, and may include other forms of vacuum control alternative to vent hole 42, such as, buttons, pressure sensitive, electronic, etc.

Handle 18 includes a pair of diametrically opposed finger notches 42 that form a substantially rectangular cross-section. This configuration facilitates gripping of apparatus 10 during rotation of the instrument about longitudinal axis 20. It is envisioned that the portion of handle 18 adjacent finger notches 42 may have various cross-sectional configurations, such as, for example, cylindrical, elliptical, polygonal, etc.

Handle 18 has an outer surface 22 including a plurality of longitudinal fins 24 extending radially therefrom. Fins 24 extend longitudinally the length of handle 18 and taper at the proximal portion of handle 18. It is contemplated that fins 24 may extend various lengths along handle 18 according to the particular requirements of an application. It is further contemplated that handle 18 may include one or a plurality of fins 24. Fins 24 desirably have a thickness in the range of 0.060-0.065 inches. It is envisioned that fins 24 may have various thickness. Fins 24 may also be variously configured such as, for example, staggered, undulating, serrated, offset, non-uniform and may include openings or cavities.

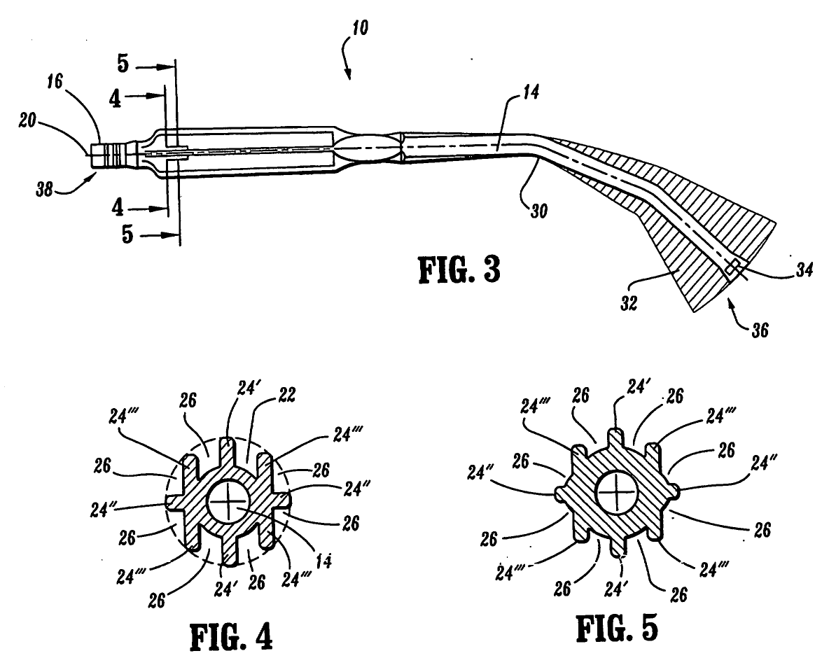

Referring to FIGS. 3-5, the plurality of longitudinal fins 24 include a first fin pair 24′ and a second fin pair 24″ that project radially outward from outer surface 22. Fins 24 of each pair 24′, 24″ are diametrically opposed about outer surface 22. First fin pair 24′ and second fin pair 24″ are disposed in a substantially relative perpendicular orientation and offset 90° relative to longitudinal axis 20 of handle 18. It is contemplated that fin pairs 24′, 24″ may be offset at various angular orientations according to the particular application requirements.

The plurality of longitudinal fins 24 further includes a third fin pair 24′″ and a fourth fin pair 24′″. Fin pairs 24′″ are opposed about outer surface 22 and disposed in alternate planes tangential to outer surface 22. The tangential planes of fin pairs 24′″ are substantially parallel. It is contemplated that the tangential planes may be alternately configured, such as, for example, disposed at a particular angular orientation, converging, diverging, etc. It is further contemplated that fins 24 may be variously disposed about outer surface 22, for example, equidistantly spaced, radially disposed at equidistant angles, etc. It is envisioned that fins 24 may be disposed in non-pair arrangements such that the fins are not opposed.

Fins 24 facilitate manipulation of apparatus 10 and define grooves 26 therebetween, that advantageously guide fluids away from handle 18. This configuration reduces fluid buildup adjacent handle 18 during a procedure and prevents potentially hazardous conditions. Reduced fluid buildup facilitates enhanced grip force and control of handle 18 and apparatus 10, as will be discussed.

Each pair of adjacent longitudinal fins 24 define a groove 26 therebetween. Grooves 26 define guide channels 28 that guide the flow of bodily fluids away from handle 18. As shown in FIG. 4, taken along cross-section line 4-4 of handle 18 in FIG. 3, fin pairs 24′, 24″, 24′″ define alternatively sized and configured grooves 26. Handle 18 has a first wall thickness t′ and grooves 26 define a corresponding volume according to the orientation of the adjacent fins 24. For example, adjacent grooves 26 may define alternate volumes, such as the volume of groove 26 (defined by fin 24″ and fin 24′″) being greater than groove 26 (defined by fin 24″ and fin 24′″). It is envisioned that grooves may alternatively define uniform volumes.

As shown in FIG. 5, taken along cross-section line 5-5 of handle 18 in FIG. 3, which is disposed distal to line 4-4 on handle 18, the wall thickness of handle 18 smoothly increases to a second wall thickness t″. The increase in the wall thickness of handle 18 correspondingly reduces the volume of grooves 26. This configuration defines a distal to proximal slope of outer surface 22. The sloping configuration directs fluid in a proximal direction from handle 18 through guide channels 28. Grooves 26 are open at the proximal end of handle 18 to allow fluid to freely drain from handle 18. It is envisioned that the radial dimension of fins 24 may increase, decrease or remain uniform along any change in wall thickness of handle 18.

It is contemplated that the wall thickness of handle 18 may have varying degrees of slope or alternatively have a stepped configuration. It is further contemplated that the wall thickness of handle 18 may be uniform or slope from a proximal to distal direction. It is envisioned that the distal ends of longitudinal fins 24 can abut the finger notch 42 portion of handle 18, or alternatively may be disposed proximal from the distal end to define a gap (not shown) for fluid drainage between longitudinal fins 24 and finger notch 42.

Adjacent fins 24 define grooves 26, which reduce the overall material requirements and weight of apparatus 10. The reduction in material of handle 18 disposes the center of mass of apparatus 10 to a position distal of handle 18. Therefore, the balance point of apparatus 10, such as, for example, the center of mass, relative to the proximal and distal end of apparatus 10, is disposed distal of handle 18. This configuration advantageously facilitates improved control of apparatus 10 during use. It is contemplated that the balance point may be disposed at various positions along the length of apparatus 10, according to the particular requirements of a medical application.

The components of apparatus 10 are fabricated from materials suitable for medical applications, such as, for example, polymerics or metals, such as stainless steel, depending on the particular catheter application and/or preference of a practitioner. Semi-rigid and rigid polymerics are contemplated for fabrication, as well as resilient materials, such as molded medical grade polypropylene. One skilled in the art, however, will realize that other materials and fabrication methods suitable for assembly and manufacture, in accordance with the present disclosure, also would be appropriate.

Apparatus 10, as shown in FIGS. 1-5 and similar to that described, is assembled, sterilized and packaged for use. For example, in a suction procedure, apparatus 10 is prepared for aspiration of fluids from a body (not shown) in contemplation of a medical procedure. Handle 18 is manipulated such that nozzle 16 aspirates fluid.

During the procedure, fluid builds up about outer surface 22 of handle 18. Fins 24 cause the fluid to become disposed within grooves 26. Guide channels 28 guide the flow of bodily fluids away from handle 18, in a distal to proximal direction, due to the sloping configuration of outer surface 22, as discussed. Accordingly, fluid is caused to drain through the openings of grooves 26 at the proximal end of handle 18. This configuration reduces fluid buildup adjacent handle 18 during a procedure and prevents potentially hazardous conditions. Reduced fluid buildup facilitates enhanced grip force and control of handle 18 and apparatus 10. Other procedures employing apparatus 10 are also contemplated including those requiring irrigation, pressurized air, etc.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A surgical instrument comprising:
a handle defining a longitudinal axis, the handle having an outer surface including a plurality of longitudinal fins that define a plurality of longitudinal grooves therebetween.

2. The surgical instrument of claim 1, further comprising an elongated tubular portion having an opening configured for suction.

3. A surgical instrument as recited in claim 1, wherein at least one of the fins project radially from the outer surface.

4. A surgical instrument as recited in claim 1, wherein a pair of the fins project radially from the outer surface and are diametrically opposed.

5. A surgical instrument as recited in claim 1, wherein a pair of the fins are opposed and disposed in a plane tangential to the outer surface of the handle.

6. A surgical instrument as recited in claim 1, wherein two separate pairs of the fins project radially from the outer surface and are diametrically disposed.

7. A surgical instrument as recited in claim 6, wherein the two separate pairs are offset 90° relative to the longitudinal axis.

8. A surgical instrument as recited in claim 1, wherein two separate pairs of the fins are opposed and disposed in alternate planes tangential to the outer surface of the handle.

9. A surgical instrument as recited in claim 1, wherein the grooves include guide channels that direct fluid to a proximal end of the handle.

10. A surgical instrument as recited in claim 1, wherein at least one groove defines a greater volume than an adjacent groove.

11. A surgical instrument as recited in claim 2, wherein the tubular portion includes a passageway that extends to the opening having a nozzle.

12. A surgical instrument as recited in claim 2, wherein the tubular portion has a curvature adjacent a distal portion thereof.

13. A surgical instrument as recited in claim 2, wherein a proximal end of the handle has an attachment configured to communicate with a suction source.

14. A surgical instrument as recited in claim 1, wherein the surgical instrument has a center of mass located distal to the handle.

15. A surgical instrument as recited in claim 1, wherein the handle has a first wall thickness that smoothly increases to a second wall thickness.

16. A surgical instrument as recited in claim 15, wherein the first wall thickness and the second wall thickness define a distal to proximal slope.

17. A surgical instrument as recited in claim 1, wherein the handle has a slope configuration that defines a distal to proximal flow direction.

18. A surgical instrument as recited in claim 1, wherein the fins are configured to facilitate gripping.

19. A surgical instrument as recited in claim 1, wherein each of the fins has a thickness in the range of 0.060-0.065 inches.

20. A medical suction apparatus comprising:
an elongated tubular portion including a passageway that extends to an opening having a nozzle that is configured for suction; and
a handle mounted with the tubular portion and defining a longitudinal axis, the handle having an outer surface including a plurality of longitudinal fins, wherein the plurality of longitudinal fins include a first and second pair of the fins that project radially from the outer surface, the fins of each pair being diametrically opposed, the first and second pairs being offset 90° relative to the longitudinal axis of the handle,
the plurality of longitudinal fins further including a third and fourth separate pair of the fins that are opposed and disposed in alternate planes tangential to the outer surface of the handle,
the plurality of longitudinal fins defining a plurality of longitudinal grooves therebetween, the grooves including guide channels that direct fluid to a proximal end of the handle.
